(19) United States
(12) Patent Application Publication
(10) Pub. No.: US 2021/0369915 A1
(43) Pub. Date: Dec. 2, 2021

(54) TRIPLE-NETWORK HYDROGEL IMPLANTS FOR REPAIR OF CARTILAGE

(71) Applicant: Duke University, Durham, NC (US)

(72) Inventors: Benjamin Wiley, Durham, NC (US); Feichen Yang, Durham, NC (US); Kenneth Gall, Durham, NC (US); Jonathan Riboh, Durham, NC (US)

(21) Appl. No.: 16/332,574

(22) PCT Filed: Nov. 7, 2018
(86) PCT No.: PCT/US18/59563

§ 371 Date: Mar. 12, 2019

Related U.S. Application Data
(60) Provisional application No. 62/699,991, filed on Jul. 18, 2018.
(60) Provisional application No. 62/582,505, filed on Nov. 7, 2017.

(51) Int. Cl. A61L 27/32; A61L 27/26; A61L 27/52; A61L 27/54; C08L 41/00; C08L 33/26

(52) U.S. Cl. CPC A61L 27/32; A61L 27/26; A61L 27/52; A61L 27/54; A61L 2430/06; C08L 33/26; A61L 2300/414; A61L 2400/12; C08L 41/00

(57) ABSTRACT

Artificial cartilage materials for repair and replacement of cartilage (e.g., load-bearing, articular cartilage). The artificial cartilage materials described herein include triple-network hydrogels including a cross-linked fiber network (e.g., a bacterial cellulose nanofiber network) and a double-network hydrogel (e.g., a double-network hydrogel including polfacrylamide-methyl propyl sulfonic acid). The artificial cartilage may be coated onto or formed into an implant (e.g., plug). The artificial cartilage may include a surface macroporosity, e.g., 0.1-300 micrometers diameter. Also described herein are methods of forming and methods of using the triple-network hydrogel artificial cartilage materials.

### CROSS REFERENCE TO RELATED APPLICATIONS

This patent application claims priority to U.S. provisional patent application No. 62/582,505 (“Tunable, Ultrastrong Hydrogels and Methods of Making and Using Same”) filed on Nov. 7, 2017 and U.S. provisional patent application No. 62/699,991 (“Devices for Cartilage Repair and Methods of Making and Using Same”) filed on Jul. 18, 2018.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates generally to triple-network hydrogel implants suitable for repair of cartilage, including specifically triple-network hydrogel joint implants and various tools, devices, systems, and methods related thereto.

### BACKGROUND

Articular cartilage lesions have limited intrinsic ability to heal, and are often associated with joint pain and chronic disability. Current strategies for cartilage restoration, including bone marrow stimulation, cartilage cell implantation, and osteochondral transplantation, have high failure rates (e.g., ˜50% at 10 years), prolonged rehabilitation times (e.g., 12-18 months), and can be very costly. A recently approved acellular hydrogel implant for treating arthritis of the big toe can reduce recovery times from six months to six weeks, but current hydrogels do not have sufficient strength to serve as a cartilage replacement in the knee and other load-bearing regions. There is a need for cartilage replacement materials and repair methods that provide immediate clinical benefit, allows immediate weight bearing, has short recovery times, and is able to fully replace the mechanical properties of hyaline cartilage for 10+ years and with low (<10%) failure rates.

### SUMMARY OF THE DISCLOSURE

In general, described herein are artificial cartilage materials for repair and replacement of cartilage (and particularly for load-bearing, articular cartilage). The artificial cartilage materials described herein typically include triple-network hydrogels having a cross-linked fiber network (e.g., a bacterial cellulose nanofiber network) and a double-network hydrogel (e.g., a double-network hydrogel including polfacrylamide-methyl propyl sulfonic acid or PAMPS in one or both networks of the double-hydrogel network). The artificial cartilage may be coated onto or formed into an implant (e.g., plug). The artificial cartilage may be configured to include a surface macroporosity, e.g., 0.1-300 micrometers diameter.

An artificial cartilage material as described herein may include a triple-network hydrogel; the triple-network hydrogel may include: a cross-linked nanofiber network having a tensile strength of greater than 5 MPa; and a double network hydrogel having a compression strength of greater than 14 MPa, wherein the cross-linked nanofiber network is between 2-25 weight % of the triple-network hydrogel.

For example, an artificial cartilage material may comprise a triple-network hydrogel including: a cross-linked cellulose nanofiber network having a tensile strength of greater than 5 MPa and a tensile modulus of greater than 8 MPa; and a double network hydrogel having a compression strength of greater than 14 MPa, wherein the cross-linked nanofiber network is between 2-25 weight % of the triple-network hydrogel.

For example, an artificial cartilage material may comprise a triple-network hydrogel including: a cross-linked bacterial cellulose nanofiber network having a tensile strength of greater than 5 MPa and a tensile modulus of greater than 8 MPa; and a negatively charged double network hydrogel including polfacrylamide-methyl propyl sulfonic acid and having a compression strength of greater than 14 MPa, wherein the cross-linked nanofiber network is between 2-25 weight % of the triple-network hydrogel.

Any of the artificial cartilage materials described herein may further include, e.g., have a shape in which, at least an outer region having a porosity of between 0.1-300 micrometers diameter. The outer region may have a thickness of between 0.1 and 2.5 mm. The artificial cartilage material may also include one or more coatings, including coatings to increase ingrowth, such as a coating on the outer region of one or more of: hydroxyapatite (HA) and insulin-like growth factor I (IGF).

In some variations, the cross-linked cellulose nanofiber network of the triple-network hydrogel comprises bacterial cellulose (BC) having a tensile modulus of greater than 8 MPa. The bacterial cellulose may be used by itself or in combination with one or more additional materials.

The triple-network hydrogels described herein may be configured to have a tensile strength of between 4-10 MPa, a tensile modulus of between 8-25 MPa, a compression strength of between 20-60 MPa, and a compression modulus of between 8-22 MPa. In some variations, the triple-network hydrogel has a coefficient of friction of less than 0.1 at 1 mm/sec.

The double network hydrogel component of the artificial cartilage material may be any double-network hydrogel having the desired compressive strength, even if the tensile strength of the double-network is lower than, e.g., 5 MPa. In particular any of the double-network hydrogels described herein may include (in one or both networks of the double-network hydrogel), a polfacrylamide-methyl propyl sulfonic acid (e.g., poly-(2-acrylamido-2-methylpropanesulfonic acid) or PAMPS). In some variations, the double network hydrogel includes a polfacrylamide-methyl propyl sulfonic acid (PAMPS) and one or more of: polyacrylamide (PAAm) and poly-(N,N′-dimethyl acrylamide) (PDMAAm).

The artificial cartilage materials described herein may be used to resurface a joint, and/or cover an implant. Thus, the artificial cartilage material may be formed into any shape or size desired. In particular, the artificial cartilage material may be formed into a plug, disk, mushroom-shape, cylinder, etc. of triple-network hydrogel.

As mentioned above, any of the artificial cartilage materials described herein, or at least an outer surface of the material, may be treated to form pores in the material. For example, the artificial cartilage material may include an outer region having a porosity of between 0.1-300 micrometers diameter. The pores may be formed by including a dissolvable material in all or a portion of the triple-network hydrogel (e.g., in an outer region of the triple-network hydrogel) as it is formed, and dissolving the material to leave pores behind. Thus the density of pores may be controlled, as well as the locations of the pores. In some variations the implant, including any pores, or exclusively in the pores, may include a material to help ingrowth of tissue, such as one or more of: hydroxyapatite (HA) and insulin-like growth factor I (IGF).

As one example, a triple-network hydrogels described herein may be formed of a material such as a triple-network hydrogel of BC-PAMPS-PAAm in which there is between about 5% and 15% (e.g., about 8%, about 9%, about 10%, about 11%, about 12%, etc.) of BC weight %.

Also described herein are methods of treating a patient using any of the artificial cartilage materials described herein, e.g., to repair or replace cartilage, including resurfacing. A method of repairing or replacing a cartilage in a subject with any of the triple-network hydrogels described herein may include implanting or inserting a body formed at least in part of a triple-network hydrogel as described herein. In some variations the body may be adhesively secured to the patient's tissue. Alternatively or additionally the body may be secured by a fixation device such as a screw, staple, suture, etc. For example, the body may be formed of a metal and/or polymeric material to which the triple-network hydrogel is attached (coated, encapsulating, affixed, etc.), and the body may be secured via a screw, pin, staple, suture, etc. to the bone and/or cartridge. Any of these methods may optionally include preparing the body region (e.g., bone, existing cartilage, etc.) by, e.g., removing tissue and/or forming a receiving region.

Any of these methods may be used treat a patient by repairing or replacing cartilage in a load-bearing joint, such as a knee, wrist, ankle, shoulder, spine, hip, etc. Alternatively and of the methods may be used to repair a non-load bearing region of the body (e.g., toe, fingers, etc.).

Also described herein are methods of forming and methods of using the triple-network hydrogel artificial cartilage materials. For example, a method of forming a triple-network hydrogel may comprise first forming a cross-linked network of nanofibers, such as bacterial cellulose (BC), or in some variations a network of bacterial cellulose and polyacrylamide (BC-PAAm), then adding the double-network hydrogel to the cross-linked network.

For example, a triple-network hydrogel may be formed by impregnating the network of nanofibers (e.g., a bacterial cellulose, such as a body, sheet, plug, etc. formed of bacterial cellulose) with the components of the first hydrogel network of the double network hydrogel. For example, the nanofibers may be soaked in a solution of monomer, cross-linker and activator in a desired amount (e.g., AMPS, MBAA and 12959) for a soaking period (e.g., overnight) and formed into a desired shape (e.g., molded, etc.) then cured, e.g., by UV curing, which may cross-link the nanofibers and/or form the first hydrogel network. After curing, the cross-linked network with the first hydrogel network may then be impregnated with the materials for forming the second network, e.g., monomers, cross-linker and activator (e.g., acrylamide, MBAA and 12959), and curved (e.g., via UV light) again to form the second hydrogel network and thus the triple-network hydrogel.

As mentioned above, in some variations, pores may be added to the material, either the entire material, or a region of the material. For example, pores of a predetermined size and/or density may be formed by adding a dissolvable material to triple-network hydrogel, or to a region of the triple-network hydrogel (e.g., the outer region). In some variations a second layer of triple-network hydrogel may be formed onto a core and the pore-forming material (e.g., calcium carbonate sand particles) may be molded around the solid hydrogel core. The dissolvable material may then be dissolved in a solvent (e.g., calcium carbonate may be dissolved in hydrochloric acid) to obtain the porous gel surface.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a table illustrating and comparing mechanical properties of articular cartilage, one example of a triple-network hydrogel for use in repairing cartilage and various possible components (e.g., cross-linked fiber networks and/or double-network hydrogels) that may be used.

FIG. 2A shows compressive stress-strain curves of triple-network hydrogels (BC-PAMPS-PDMAAm hydrogels) having different concentrations of BD (e.g., 1.7 weight % BC and 6 weight % BC).

FIG. 2B shows tensile stress-strain curves of triple-network hydrogels (BC-PAMPS-PDMAAm hydrogels) having different concentrations of BD (e.g., 1.7 weight % BC and 6 weight % BC).

FIG. 3 is a graph showing the apparent strain curve for a material such as cartilage (or many of the triple-network hydrogels that may be used as an artificial cartilage).

FIG. 4A shows tensile stress-strain curves of BC-PAMPS-PAAm hydrogels with different concentrations of BC.

FIG. 4B shows tensile stress-strain curves of CNF-PAMPS-PAAm with different concentrations of CNF.

FIG. 5A shows compressive stress-strain curves of BC-PAMPS-PAAm hydrogel with different concentrations of BC.

FIG. 5B shows compressive stress-strain curves of CNF-PAMPS-PAAm with different concentrations of CNF.

FIG. 6A is a table (table 2) showing a comparison of cellulose type and concentration for tensile strength and Young's modulus at 10% strain of various triple-network hydrogels in which the cross-linked fiber network is cellulose of either BC or CNF type.

FIG. 6B is a table (table 3) showing a comparison of cellulose type and concentration for compression strength and Young's modulus at 25% strain of various triple-network hydrogels in which the cross-linked fiber network is cellulose of either BC or CNF type.

FIG. 7A is a graph showing the coefficient of friction of cartilage and of a synthetic cartilage formed of a triple-network hydrogel as described herein.

FIG. 7B is a table (table 4) comparing the coefficient of friction of a native cartilage and an exemplary triple-network hydrogel (e.g., BC-PCAMPS-PAAm).

FIGS. 8A-8D illustrate one method of attaching a triple-network hydrogel to a patient's tissue.

FIG. 9 is a table (table 5) illustrating parameters that may be modified within a range to tune the mechanical parameters of an exemplary triple-network hydrogel (e.g., BC-PCAMPS-PAAm).

FIG. 10 is a table (table 6) illustrating examples of parameters (surface porosity thickness, types of surface coatings, e.g., HA, IGF) that may be modified in any of the triple-network hydrogels described herein.

FIGS. 11A-11D illustrate one example of a triple-network hydrogel including a macroporous surface (and an internal porosity that is microporous). FIG. 11A shows the implant with a porous outer surface; in FIG. 11B a liquid material (e.g., blood) has been added in contact with the outer surface; in FIG. 11C the liquid material is shown wicked through the pores of the outer surface; and FIG. 11D shows that the inner, microporous region, is not appreciably infiltrated by the blood.

FIGS. 12A-12C illustrate an example of a method of using a triple-network hydrogel to repair cartilage. In FIG. 12A, a region of bone includes a missing region of cartilage (and/or bone and cartilage, as shown). The missing region may be surgically created or modified, e.g., from a modified defect in the bone. A triple-network hydrogel may be added to fill the defect, as shown in FIG. 12B. FIG. 12C shows an example in which the triple-network hydrogel includes a porous outer region (pores not shown to scale or representative density).

### DETAILED DESCRIPTION

The methods, materials and apparatuses including them (including implants) described herein relate generally to triple-network hydrogels, and particularly those including a cross-linked fiber (e.g., nanofiber) network having a tensile strength that is greater than about 5 MPa and a tensile modulus of greater than about 5 MPa (e.g., between about 5-25 MPa), combined with a double-network hydrogel having a compressive strength of greater than about 24 MPa and a compression modulus of between about 10-20 MPa. The combination of the cross-linked fiber network and the double-network hydrogel is a triple-network hydrogel material. The materials and methods may provide, in part, tunable, ultrastrong hydrogels that may have substantially the same time-zero mechanical properties (or superior properties) as cartilage and the capability for tissue ingrowth and integration.

These triple-network hydrogel compositions may be used to treat a subject in need, for example, for articular cartilage replacement applications that meet required mechanical strength to withstand high loads of human joints. The triple-network hydrogels provided herein can be used in a body to augment or replace any tissue such as cartilage, muscle, breast tissue, nucleus pulposus of the intervertebral disc, other soft tissue, interpositional devices that generally serves as a cushion within a joint, etc.

The triple-network hydrogel compositions described herein may comprise, consists of, or consists essentially of: (i) a cross-linked fiber network; and (ii) a double network hydrogel with compressive strength of greater than about 20 (e.g., greater than about 22, greater than about 23, greater than about 24, greater than about 25, between about 20 and 60, between about 22 and 55, between about 23 and 50, between about 24 and 46, etc.) and a compressive modulus of greater than about 8 MPa (e.g., greater than about 9 MPa, greater than about 10 MPa, between about 8-25 MPa, between about 9-22 MPa, between about 10-20 MPa, etc.). The double network hydrogel may be negatively charged.

The cross-linked fiber network and the double-network hydrogels forming the triple-network hydrogel compositions described herein may be selected based on their mechanical properties. Any appropriate double-network hydrogel and/or cross-linked fiber network having the specified mechanical properties may be used. For example, the triple-network hydrogel compositions described herein may comprise, consists of, or consists essentially: (i) a cross-linked fiber network having a tensile modulus of greater than about 5 MPa (e.g., greater than about 8 MPa, greater than about 8.2 MPa, greater than about 8.4 MPa, between about 5 MPa and about 25 MPa, between about 8 MPa and about 30 MPa, between about 8 MPa and about 25 MPa, between about 8.4 MPa and about 23 MPa, etc.) and tensile strength of greater than about 5 MPa (e.g., greater than about 4 MPa, greater than about 5 MPa, greater than about 5.2 MPa, between 4-20 MPa, between about 4.5-10 MPa, between about 5-9 MPa, etc.); and (ii) a double network hydrogel (e.g., a negatively charged double-network hydrogel) with a compressive strength of greater than about 13 MPa (e.g., greater than about 14 MPa, greater than about 20 MPa, greater than about 22 MPa, greater than about 23 MPa, greater than about 24 MPa, greater than about 25 MPa, between about 13-65 MPa, between about 14-59 MPa, between about 20 and 60 MPa, between about 22 and 55 MPa, between about 23 and 50 MPa, between about 24 and 46 MPa, etc.). In some variations the double-network hydrogel may have a compressive modulus (e.g., equilibrium modulus) of greater than about 8 MPa (e.g., greater than about 9 MPa, greater than about 10 MPa, between about 8-25 MPa, between about 9-22 MPa, between about 10-20 MPa, etc.).

The cross-linked fiber network and the double network hydrogel may be included in the triple-linked network in any appropriate percentage (e.g., weight %). For example, the triple-linked network may include between 2-20% weight % (e.g., between about 2%, 3%, 4%, 5%, 6%, 7%, 8%, 9%, 10%, etc. and about 15%, 16%, 17%, 18%, 19%, 20%, 21%, 22%, 23%, 24%, 25%, etc.) of the cross-linked fiber network and the double-network hydrogel may be between 75-98% weight %. The final percentages may be tuned specifically to the components (e.g., the particular double-network hydrogel and/or cross-linked fiber network), the body region, the patient and/or the cartilage being replaced by the implant.

The cross-linked fiber networks described herein may be any appropriate cross-linked fiber network. The cross-linked fiber network is biocompatible, and may be cross-linked covalently or via hydrogen bonding. In some variations the cross-linked fiber network is a cross-linked nanofiber. One non-limiting example of a cross-linked network is a cross-linked nanofiber cellulose network. The fiber network may be, for example, bacterial cellulose (BC), or in some variations a network of bacterial cellulose and polyacrylamide (BC-PAAm). For example, the tensile strength of BC-PAAm is may be greater than 5 MPa (e.g., between 30-50 MPa, or about 40 MPa), and the tensile modulus may be greater than 5 MPa (e.g., up to between 100-120 MPa). The tensile strength and modulus may depend, at least in part, on the density of the bacterial cellulose. The compressive strength of BC-PAAm is relatively poor (e.g., about 5.1 MPa). In addition to BC-PAAm, other cross-linked fiber networks may be use instead (or in addition to). For example, other cross-linked fiber networks may include electrospun poly(vinyl alcohol) (PVA) fibers, aramid nanofibers (e.g., Aramid-PVA nanofibers), wet-spun silk protein fiber, chemically crosslinked cellulose nanofiber, polycaprolactone fibers (e.g., 3D woven PCL fibers), electrospun gelatin nanofibers, etc., any of which may be adjusted so that the tensile strength is within the desired range (e.g., greater than 5 MPa with a tensile modulus of >8 MPa, etc.).

The double-network hydrogels used as part of the triple-network hydrogels described herein may be any appropriate double-network hydrogel, particularly those having the desired mechanical properties (e.g., compressive strength). In general, the double-network hydrogel is biocompatible. The double-network hydrogel typically includes two networks having non-identical properties. For example, the first network can be stiff and/or brittle and can be cross-linked (e.g., photo cross-linked) with a second network that is soft and/or ductile. The multi- or dual network hydrogel may then have properties, including compression strength and modulus, that are non-identical to those of the individual networks alone. For example, while the first network alone may be too brittle for use as a load bearing implant and the second network may be too soft, the two networks, when combined to form the present hydrogels, may possess the structural, mechanical, and biological characteristics required. For example, the double-network hydrogels can have an internal structure with desirable mechanical properties suitable for use as part of the triple-network hydrogels described herein. The precise mechanical properties of the double-network hydrogel can be altered by varying the ratio of the polymer in the first network to that of the polymer in the second network. Alternatively, or in addition, one can vary the crosslinking densities.

For example, a double-network hydrogel may be a poly-(2-acrylamido-2-methylpropanesulfonic acid) (PAMPS) based double-network hydrogel, such as a PAMPS and poly-(N,N′-dimethyl acrylamide) (PDMAAm) double-network gel. A PAMPS-PDMAAm double-network hydrogel may have excellent biocompatibility and resistance to biodegradation, particularly when combined with a cross-linked fiber network (such as BC-PAAm) to form a triple-network hydrogel. For example, the compression strength of a PAMPS-PDMAAm double-network hydrogel may be equal to or greater than about 14 MPa (e.g., greater than 15 MPa, greater than 18 mPA, greater than 20 MPa, greater than 22 MPa, etc.), although the modulus of compression is typically very low (e.g., approximately 0.33 MPa) as is the tensile strength and modulus. In some variations the double-network hydrogel may be itself negatively charged, or it may include an agent to make it negatively charged. For example PAMPS-PDMAAm typically has a negative charge density (mEq/mL).

Other double-network hydrogels having the appropriate mechanical property (e.g., compression strength and/or charge and/or friction coefficient and/or wear resistance) may be used. These include those produced by copolymerization of 1-vinylimidazole and methacrylic acid, double-network hydrogels based on amphiphilic triblock copolymers, polyampholyte hydrogels, a PVA-tannic acid hydrogel, a poly(N-acryloyl) glycinamide hydrogel, polyacrylic acid-acrylamide-C18 hydrogel, Guanine-boric acid reinforced PDMAA, polyelectrolyte hydrogels, a poly(acrylonitrile-co-1-vinylimidazole) hydrogel (e.g., a mineralized poly(acrylonitrile-co-1-vinylimidazole) hydrogel), a PAMPS/MMT clay composite hydrogel, a polyacrylic acid-Fe3+-chitosan hydrogel, a PMAAc gel, a Graphene oxide/Xonotlite reinforced PAAm gel, a poly(stearyl methacrylate)-polyacrylic acid gel, an annealed PVA-PAA hydrogel, supramolecular hydrogels from multiurea linkage segmented copolymers, PAN-PAAm hydrogel, a microsilica reinforced DMA gel, a Agar-PHEMA gel.

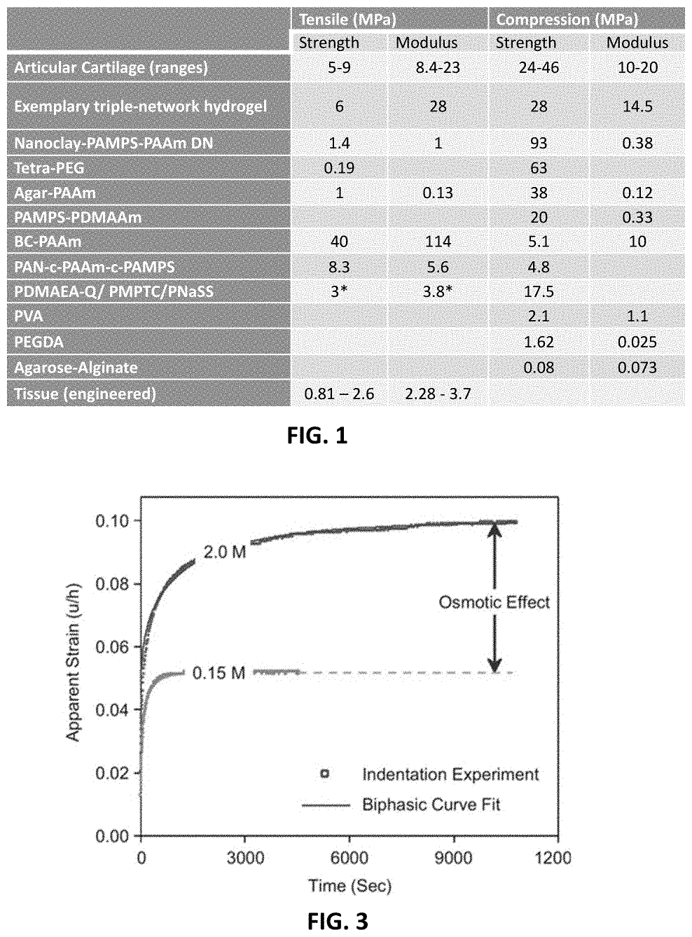

Examples of suitable materials for the hydrogel are provided in FIG. 1, showing a table include the mechanical properties of articular cartilage and an exemplary triple-network hydrogel, as well as listing and providing properties of possible double-network hydrogels and cross-linked fibers (e.g., nanofibers) some of which may be used to form the triple-network hydrogels described herein. In FIG. 1, materials listed include PAMPS (polfacrylamide-methyl propyl sulfonic acid); PAAm (polyacrylamide); PAA (polyacrylic acid); PVA (polyvinyl alcohol); PEG (polyethylene glycol); CTAB (Cetyl trimethylammonium bromide); PNIPAM (Poly(N-isopropylacrylamide)); PDAAm (polydimethylacrylamide); PDAEA-Q (polyfacryloyloethyltrimethylammonium chloride); PMPTC (poly(3-(methylacryloylamino)propyl-trimethylammonium chloride); PNaSS (poly(sodium p-styrenbesulfonate)); BC (bacterial cellulose); PAN (polyacrylonitrile); c (copolymer); PFGDA (polyethylene glycol diacrylate); PEG (polyethylene glycol).

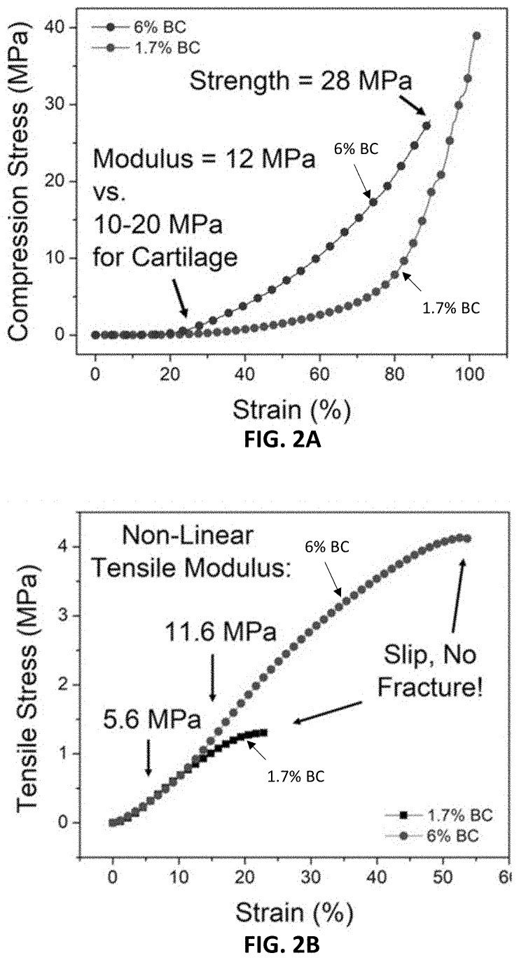

The compositions described herein may combine the excellent tensile strength of cross-linked (e.g., nanofiber) networks with the excellent compression strength of a double-network hydrogel such as a PAMPS-based hydrogel to create a hydrogel that has the tensile and compressive strength of cartilage. This results in a triple-network hydrogel material that mimics mechanical properties of and structure of cartilage, which consists of a large fraction (e.g., up to 22%) of strong, cross-linked collagen nanofibers, and a negatively charged matrix. In some such triple-network structures, the collagen is replaced by cellulose nanofibers, and the negative charge comes from the PAMPS double network hydrogel. In certain embodiments, the hydrogel comprises a PAMPS-PDMAAm hydrogel. For example, FIGS. 2A-2B illustrates mechanical properties of a triple-network hydrogel that is formed of PAMPS-PDMAAm double-network hydrogel and a cross-linked bacterial cellulose (BC) nanofiber network (at 6 weight %), resulting in a material having exceptional biocompatibility and resistance to biodegradation.

As shown in FIG. 1, aside from the exemplary triple-network hydrogel (e.g., a PAMPS-PDMAAm double-network hydrogel and a cross-linked bacterial cellulose (BC) nanofiber network (at 6 weight %)) the components, including cross-linked nanofiber networks and double-network hydrogels, separately lack both the tensile and the compression strength of cartilage. For example, a nanoclay-PAMPS-PAAm hydrogel has excellent compression strength (e.g., 93 MPa), but a relatively poor tensile modulus and tensile strength. On the other hand, a double network gel consisting of bacterial cellulose and polyacrylamide (BC-PAAm) has a very high tensile strength (up to 40 MPa) and modulus (up to 114 MPa) depending on the density of the bacterial cellulose in the gel, but relatively poor compression strength (5.1 MPa).

The triple-network hydrogels described herein combine the excellent tensile strength of cross-linked fiber (e.g., nanofiber) networks with the excellent compression strength of a PAMPS-based hydrogel to create a gel that has the tensile and compression strength of cartilage. This approach mimics the structure of cartilage, which consists of a large fraction of strong, cross-linked collagen nanofibers, and a negatively charged matrix. The triple-network hydrogel materials described herein may replace the collagen with another cross-linked fibrous network, such as cellulose nanofibers (e.g., bacterial cellulose), and the negative charge may be included by the double-network hydrogel (e.g., a PAMPS double network hydrogel). PAMPS-PDMAAm hydrogel has previously been demonstrated to have excellent biocompatibility and resistance to biodegradation. FIGS. 2A and 2B shows results from testing two different triple-network hydrogels: a PAMPS-PDMAAm double-network hydrogel and a cross-linked bacterial cellulose (BC) nanofiber network at two different weight percentages of the cross-linked bacterial cellulose (BC) nanofiber network (1.7 weight % BC and at 6 weight % BC).

In FIGS. 2A and 2B, the 6 wt. % BC triple-network hydrogel shows a compression strength, dynamic compression modulus (FIG. 2A) and a non-linear tensile modulus (FIG. 2B) approximately equivalent to cartilage.

The triple-network hydrogels described herein may also have similar or superior hydraulic permeability and fixed charge density as compared to cartilage. The water content, and thus permeability, of the components of the triple-network hydrogel (e.g., a double-network hydrogel such as PAMPS-PDMAAm) can be varied by changing the amount of monomer and cross-linker in the solution before carrying out the polymerization. The effect of the fixed charge density and thus osmotic pressure can be determined by comparing the time-dependent strain response at 0.15 M to that at 2 M, as illustrated in FIG. 3. Quantitative values of fixed charge density can be extracted from mechanical property measurements by fitting a triphasic model to the data at 0.15 M. For example, a pressure-dependent friction coefficient of the triple-network hydrogel (such as the exemplary BC-PAMPS-PDMAAm hydrogels) may be measured on a tribometer.

Thus, the triple-network hydrogels described herein may mimic key properties of cartilage. Articular cartilage principally consists of water (60-85% by weight), type II collagen fibers (15-22%) with diameters of ˜100 nm, and negatively charged Aggrecan (4-7%). The collagen nanofibers give cartilage its stiffness in response to tensile stress (stretching) and shear, whereas its resistance to compression at short time scales is primarily due to its low permeability to water. The rate of deformation under compression is typically quantified with a characteristic time constant (τ), which is defined in terms of the aggregate compressive modulus (HA, a measure of stiffness in confined compression), hydraulic permeability (k), and thickness (h): τ=h2/HAk. FIG. 3 shows that for a constant force applied during an indentation test, there is very little deformation at short time scales (<300 s), meaning that cartilage initially feels very hard when pressed. At these short times scales more than 95% of the total stress applied to cartilage is born by the interstitial fluid, giving cartilage an apparent stiffness of, e.g., 10-20 MPa and an extremely low friction coefficient. As the time of the applied force increases, the cartilage deforms and extrudes liquid until it reaches an equilibrium, at which point the apparent compressive modulus HA=0.5 MPa. This stiffness is too small to support the peak compressive stresses (e.g., 10-20 MPa) in the knee, meaning that under physiological conditions the pressure in the joint is mostly supported by pressurized fluid. However, the equilibrium modulus may determine the rate of deformation and recovery. In articular cartilage, between 30-50% of the equilibrium modulus is due to the osmotic pressure from the negatively charged aggrecan. This osmotic pressure effect can be observed in graphs such as those shown in FIG. 3, wherein the strain (deformation) increases when the concentration of salt in the electrolyte bath is increased from isotonic (0.15 M) to hypertonic (2.0 M) conditions. The hypertonic bath screens out the fixed charge on the aggrecan and removes the osmotic pressure effect.

The triple-network hydrogels described herein may have similar time-dependent mechanical properties and a low coefficient of friction equivalent to natural human cartilage. As shown in FIG. 3, these triple-network hydrogels may have a nonlinear tensile modulus similar to that exhibited by the cross-linked collagen nanofiber matrix, a low permeability to fluid flow, and a large negative fixed charge density. In addition, the triple-network hydrogel synthetic cartilage described herein may have high tensile and compressive strength so that it does not fracture. Hydrogels mostly consist of water and have a low permeability, giving them a very low coefficient of friction. However, current hydrogels do not have sufficient mechanical strength to serve as a load-bearing cartilage replacement.

Another example of a triple-network hydrogel as described herein are triple-network hydrogels formed from a double-network of a PAMPS-PAAm hydrogel and a percentage (e.g., between 1-25 weight %) of dense cross-linked fiber (e.g., nanofiber) network, such as bacterial cellulose (BC), bacterial cellulose and polyacrylamide (BC-PAAm), or cellulose nanofibers (CNF).

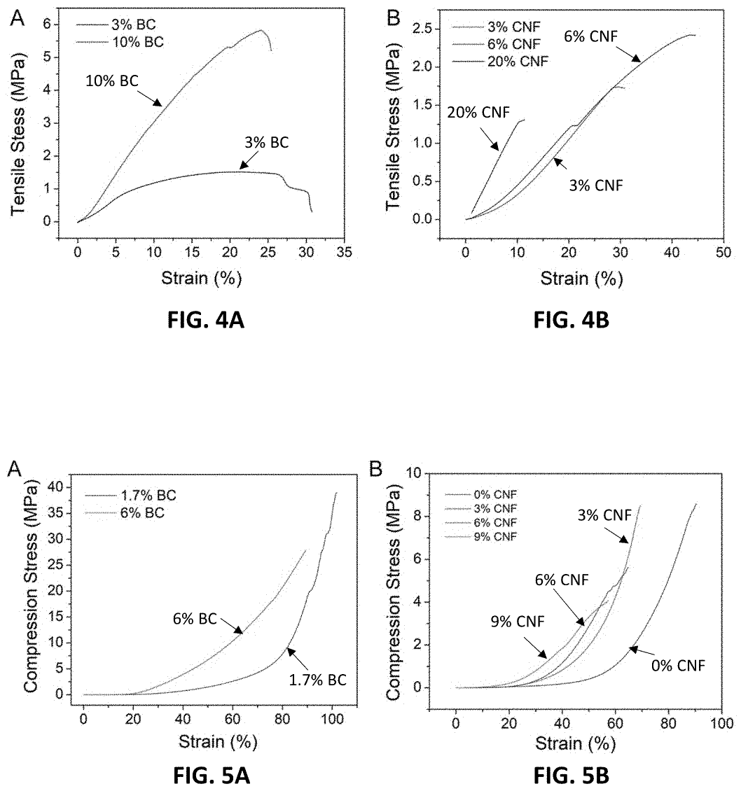

FIGS. 4A and 4B illustrate tensile strength testing of other triple-network hydrogels. In FIG. 4A, the tensile stress profiles for both a triple-network BC-PAMPS-PAAm hydrogel having 3 weight % BC and a triple-network BC-PAMPS-PAAm hydrogel having 10% BC are shown. FIG. 5B shows the tensile strength profiles for three different triple-network CNF-PAMPS-PAAm hydrogels having 3 weight % CNF, 6 weight % CNF and 20 weight % CNF, respectively.

Similarly, FIG. 5A shows a compression stress profile for a triple-network BC-PAMPS-PAAm hydrogel having 6 weight % BC and a triple-network BC-PAMPS-PAAm hydrogel having 1.7% BC. FIG. 5B shows compression stress profiles for CNF-PAMPS-PAAm hydrogels having 0 weight % CNF, 3 weight % CNF, 6 weight % CNF and 20 weight % CNF, respectively.

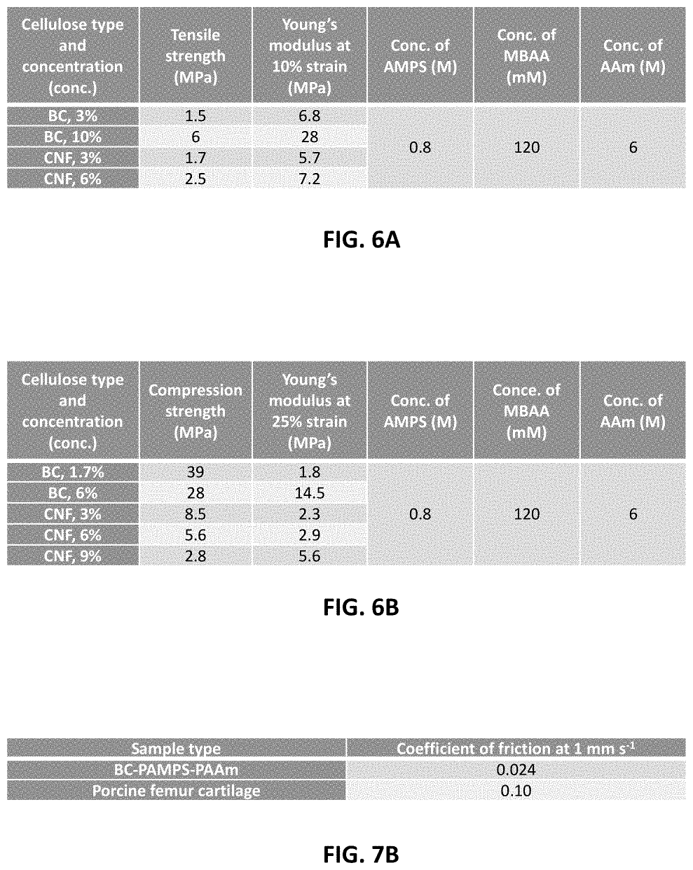

Based on Tensile test such as those shown in FIGS. 4A-5B, the mechanical properties of different BC-PAMPS-PAAm and CNF-PAMPS-PAAm samples were examined. The tensile tests were conducted with a materials tester (e.g., Instron 1321) with a shear rate of 0.25 mm/s. As shown in FIG. 4A, with an increased BC concentration from 3% to 10%, the Young's modulus of the sample at 10% strain increases from 6.8 MPa to 28 MPa. The tensile strength of the samples also increased from 1.5 MPa to 6 MPa. Comparing to the Young's modulus (5-25 MPa) and tensile strength (15-25 MPa) of cartilage, a BC-PAMPS-PAAm sample with 10% BC has the most similar tensile properties. On the other hand, shown in FIG. 4B, the maximum tensile strength that can be obtained with uncrosslinked cellulose nanofibers (CNF) is 2.5 MPa, which is far below the tensile strength of cartilage. FIG. 6A (Table 2) shows a comparison of the cellulose type and concentration for various triple-network hydrogels in which the cross-linked fiber network is cellulose of either BC or CNF type.

The mechanical compressive properties of BC-PAMPS-PAAm and CNF-PAMPS-PAAm samples were also examined. Compression tests were conducted with a materials tester (e.g., Instron 1321). As shown in FIG. 5A, with a concentration of BC of 6 wt. %, the BC-PAMPS-PAAm hydrogel has a compression strength of 28 MPa, which is comparable to cartilage (e.g., 35.7±11.25 MPa). On the other hand, if the cellulose nanofiber is not cross-linked (as with CNF), the maximum compression strength for a CNF-PAMPS-PAAm sample is 9 MPa, which is lower than the target for a cartilage replacement. Table 3 (FIG. 6B) summarizes these results.

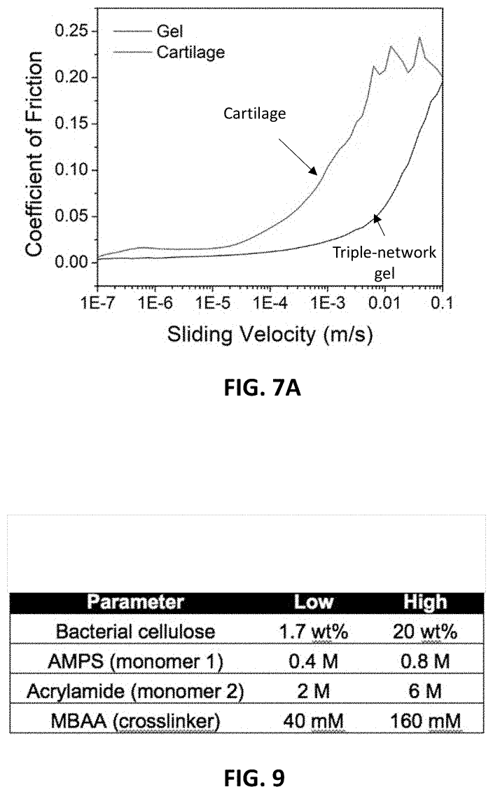

The triple-network hydrogels described herein also had other mechanical properties that were comparable (or superior to) native cartilage. For example, FIG. 7 is a graph comparing the coefficient of friction of native (e.g., articular) cartilage to an exemplary triple-network hydrogel as described herein. In FIG. 7A, the coefficient of friction of a triple-network hydrogel and cartilage against a UHMWPE surface under different sliding velocity is shown. Table 4 (FIG. 7B) summarizes the results of the tribological properties of BC-PAMPS-PAAm samples and cartilage samples, showing a lower (and therefore superior) coefficient of friction at 1 mm/s for the exemplary triple-network hydrogel tested (e.g., BC-PCAMPS-PAAm). The tribology tests were conducted on a rheometer (e.g., Anton Paar, MR302) with a tribology accessory (e.g., Cell T-BTP). The tests were run with a 3 pin-on-disk configuration. The cartilage pins were extruded from pig femur samples obtained from a local grocery store with a core extruder (e.g., Arthrex, OATS kit) with a 6 mm donor. The hydrogel pins were extruded from hydrogel sheets with the same core extruder. The sizes of pins were 6 mm×6 mm.

During the test, the 3 pins were pressed against a piece of flat ultrahigh molecule weight polyethylene (UHMWPE) disk with a controlled normal force of 15 N (0.17 MPa). 5 mL of PBS was added to act as a lubricant. The coefficient of friction was monitored within a range of sliding velocities from 10⁻⁷ m s⁻¹ to 0.1 m s⁻¹. As shown in FIG. 7A, the hydrogel sample displayed a lower coefficient of friction than the cartilage sample. The triple-network hydrogel sample showed a remarkably low coefficient of friction of 0.024 at a sliding velocity of 10⁻³ m s⁻¹, while the cartilage samples showed a much higher coefficient of friction of 0.10. This test indicates the excellent lubrication properties of our triple-network hydrogels (such as the BC-PAMPS-PAAm hydrogel).

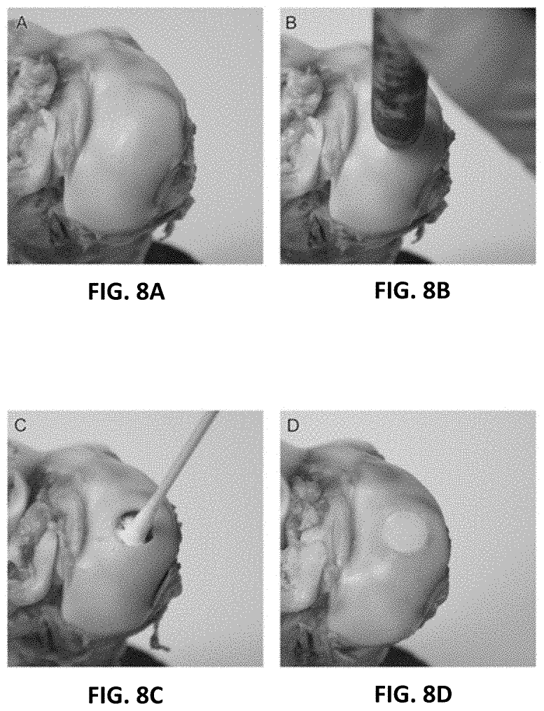

As mentioned above, any of the methods, compositions and apparatuses (e.g., implants, plugs, etc.) may be used with a biocompatible adhesive and/or attachment to an implant body formed of a material (biocompatible scaffold, body, etc.). Fixation strategies for hydrogels may include an inlay fit augmented with fibrin glue. Alternatively or additionally, an ultrastrong adhesive may be used for fixation of any of the triple-network hydrogels (e.g., to attach to bone and/or cartilage) described herein, to potentially enable weight bearing immediately after surgery, thereby accelerating recovery. A variety of tough biocompatible adhesives that have adhesion energies >1000 J m⁻², which is stronger than the adhesion energy of native cartilage to bone (800 J m⁻²). In comparison, fibrin and cyanoacrylate glues have adhesion energies of ˜10 and 100 J m⁻², respectively. For example, the tough adhesive may include a bridging polymer with primary amines (e.g. chitosan) and crosslinking agents (Sulfo-NHS & EDC) that form covalent bonds between primary amine groups and carboxylic acid groups in the hydrogel matrix and the tissue. The glue may be biocompatible, set in minutes in wet environments, and can be formulated with a UV-curable polyethylene glycol matrix. Thus, excess glue can be wiped away from the surface of the cartilage after plug insertion but before UV-curing for ˜30 seconds to ensure the interface between the plug and the cartilage is smooth and free of defects (see FIGS. 8A-8D, for example). A triple-network hydrogel may be glued into a defect in bone and/or cartilage with a biocompatible ultrastrong adhesive. In the example shown, FIG. 8A shows the original cartilage; FIG. 8B shows removal of a damaged region. FIG. 8C shows the application of biocompatible adhesive, and FIG. 8D shows insertion of the artificial cartilage (e.g., triple-network hydrogel), and removal of any excess adhesive, before a UV-cure.

Any of the triple-network hydrogels described herein may be modified to include pores. In particular, an outer thickness region of a triple-network hydrogel may be modified to include a porosity of between, for example, 0.1-300 micrometers diameters (e.g., between about 0.5-250 μm, between about 1-200 μm, etc.). The pore sizes may be selected from within a subrange of this range (e.g., between 10-200 μm, between 10-150 μm, between 10-100 μm, between 50-300 μm, between 50-200 μm, between 50-150 μm, etc.). The size range may vary or may be within a tight range (e.g., +/−50%, +/−40%, +/−30%, +/−25%, +/−20%, +/−15%, +/−10%, +/−5%, etc.). The pores maybe formed on the outer dimeter of the implant material (but not on the inner region), such as, for example on at least the outer 0.5 mm, outer 0.75 mm, outer 1 mm, outer 1.5 mm, outer 2 mm, outer 2.5 mm, outer 3 mm, etc. In some variations, the pores may be on the less than the outer 1 mm, less than the outer 1.5 mm, less than the outer 2 mm, less than the outer 2.5 mm, less than the outer 3 mm, etc. (or between about 0.25 mm and about 5 mm, between about 0.35 mm and about 4 mm, between about 0.5 mm and about 3 mm, between about 0.5 mm and 2 mm, etc.). Any appropriate density of pores may be included (e.g., between a high density of pores and a low density of pores; a high density of pores may provide a nearly continuous pathway into the implant).

The inclusion of pores may modify the triple-network hydrogel to enhance cellular infiltration and biocompatible integration with surrounding tissue in the body. Cellular infiltration may be particularly useful where the triple-network hydrogels described herein is used for cartilage resurfacing and/or replacement in a subject. The cartilage resurfacing may be performed on a weight-bearing joint. In certain embodiments, the joint comprises a knee or hip.

In some variations, the triple-network hydrogels described herein may be used with a biocompatible adhesive, such as an ultrastrong adhesive for fixation of the hydrogel to an implanting scaffold or directly to the body, e.g., to bone and cartilage to enable weight bearing immediately after surgery, thereby accelerating recovery.

Based on the limitations of biologic cartilage restoration described above, there has been a growing interest in focal joint resurfacing using durable orthopedic materials to fill chondral or osteochondral defects, such as polyethylene plugs coated with hyaluronic acid and a cobalt chrome alloy. However, these implants have limited ability to biologically integrate, and one out of five patients has to be converted to arthroplasty after an average of 4 years. Also, fixation of these devices requires mechanical anchoring to bone, potentially leading to subchondral bone deficiency if revision surgery is needed. Finally, since these implants do not match the tribology of native cartilage, there is significant concern about abnormal stress and strain distribution as well as opposing surface wear, which are known to lead to degenerative joint changes.

The methods, compositions and apparatuses described herein may combine biologic and resurfacing principles to create a more ideal cartilage replacement. The triple-network hydrogels described herein may, among other uses, provide constructs that has the same time-zero biomechanical properties as cartilage, yet retain the capability for long-term integration to surrounding bone and cartilage. Thus, these triple-network hydrogels may be used for focal joint resurfacing that has the potential to enable immediate weight bearing, short recovery times, and better long-term biocompatibility.

In some variations, a replacement material (e.g., artificial cartilage) for articular cartilage, includes a synthetic triple-network hydrogel that would ideally have at a minimum the compressive and tensile strength of cartilage, a comparable time-dependent deformation and recovery, and a very low coefficient of friction so as to resist wear over time while not causing opposing surface wear. In addition, the triple-network hydrogel may resist degradation and retain these mechanical and tribological properties over many cycles of deformation, and over many years, within the synovial fluid. Finally, the triple-network hydrogel may enable rapid integration with surrounding tissues, and long-term biocompatibility.

The triple-network hydrogels described herein having cartilage-equivalent mechanical properties maybe modified to enhance their ability to integrate with surrounding tissue, which may accelerate surgical recovery while improving implant durability. For example, in one set of triple-network hydrogels, e.g., cellulose nanofiber-reinforced double network hydrogels, a double-network (e.g., PAMPS-PDMAAm) hydrogel may be combined with a cellulose nanofiber network to obtain a triple-network hydrogel with a compressive and tensile strength comparable to cartilage; such a triple-network hydrogel may be modified to increase at least the surface (or near-surface) porosity.

For example, the triple-network hydrogel surface porosity (and/or coating) may be modified for biologic integration. For example, at least the surface of the triple-network hydrogel that is in contact with bone and cartilage may be macroporous to enable rapid integration with surrounding tissues. The bulk of the hydrogel may be nanoporous to achieve the low fluid permeability useful for cartilage-equivalent stiffness.

FIG. 9 (Table 5) illustrates some exemplary parameters of triple-network hydrogels that may be modified within ranges, including the specified ranges. The resulting triple-network hydrogel may have slightly different mechanical, fatigue, and/or wear properties. In some variations, the triple-network hydrogels described herein may also be referred to as nanofiber-reinforced double network (NR-DN) hydrogels, and they may matches the dynamic and static mechanical properties of cartilage, while minimizing the coefficient of friction so as to minimize the potential for wear.

For example, four components of a NR-DN hydrogel that may be varied to tune the exact mechanical properties (within a broader range of acceptable mechanical properties) are shown in FIG. 9 (listing the input parameters, as well as ranges of values that may be used). Variations within these triple-network hydrogels may be more or less optimized for use with a particular tissue (cartilage), body region (knee, shoulder, hip, spine, etc.), patient, etc., based on one or more of compression strength, compression modulus, tensile strength, tensile modulus, compression fatigue, tensile fatigue and coefficient of friction. Within the acceptable hydrogels, a particular hydrogel may be selected based on the mechanical, fatigue, tribological, and wear properties (all continuous variables) between solid and surface-porous constructs.

In some variations, the concentration of cross-linker may be reduced to increase the fatigue threshold. As mentioned above, the coefficient of friction between a DN gel and cartilage is lower than that between cartilage and cartilage, so most, if not all, triple-network hydrogels may exhibit acceptable coefficients of friction and wear.

In any of the triple-network hydrogels described herein, the macroporosity and chemotactic factors facilitate the integration of the implant with surrounding bone and cartilage may be adjusted. Histological analysis of the vitality of the tissue surrounding the hydrogel, as well as the glycosaminoglycan (GAG) and collagen content at the tissue-hydrogel interface of implanted triple-network hydrogel plugs indicates that porosity may enhance tissue ingrowth and biological anchoring of triple-network hydrogel implants. For example, the structure of the hydrogel-tissue interface may be altered by in-growth following implantation.

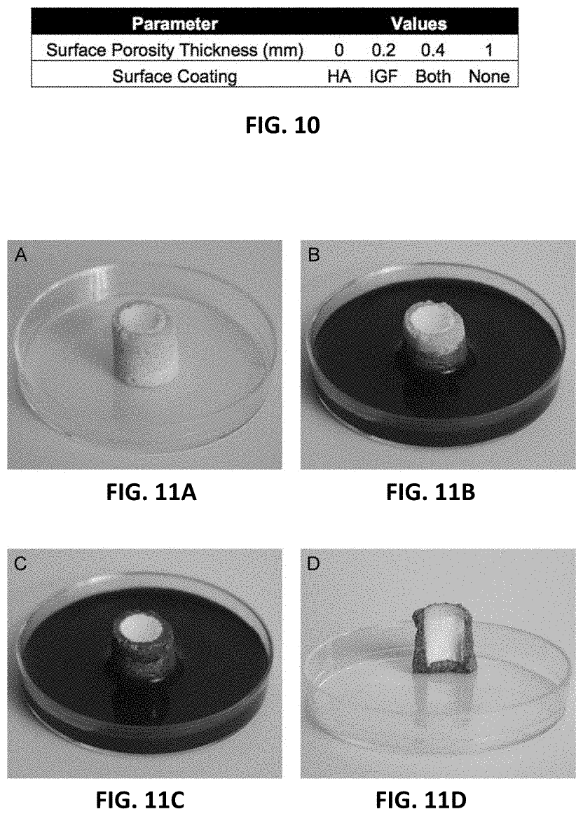

In general, the surface porosity thickness may be varied, e.g., between 0 (not porosity) to 2.5 mm thickness (e.g., 0, about 0.2, about 0.4, about 1 mm, etc.). Alternatively or additionally, a chemotactic coating may be used to create a triple-network hydrogel with a porosity on its sides and/or base. For example, in some variations a two-step molding process may be used to set porosity. Pores of a predetermined size and/or density may be formed by adding a dissolvable material to the entire triple-network hydrogel or an outer region of the triple-network hydrogel. In one example, a shell of gel containing calcium carbonate sand (e.g., particles ˜0.25 mm in diameter) may be molded around a solid gel core. The calcium carbonate may then dissolved in hydrochloric acid to obtain the porous gel surface. An example of the results of this process for one example of a gel is shown in FIGS. 11A-11D, in which simulated blood wicks into the porous shell of the gel but does not penetrate the interior. To create a chemotactic coating that stimulates bone growth, the portion of the NR-DN hydrogel that interfaces with bone may be soaked 5 times for 2 minutes in alternating solutions of dipotassium hydrogenphosphate (K₂HPO₄, 300 mM) and calcium chloride (CaCl₂, 500 mM) to form hydroxyapatite (HA) within the surface of the gel. This technique greatly improves osseointegration at 4 weeks. To improve integration with cartilage, the triple-network hydrogel may be soaked (e.g., the portion of the gel that interfaces with cartilage) with a combination of collagenase (0.6%) and insulin-like growth factor I (IGF, 25 ng/ml). This combination may promote chondrocyte repopulation of the zone of chondrocyte death in the periphery of osteochondral grafts. Both surface macroporosity and surface chemotactic coating may improve integration of tissue within the triple-network hydrogel implant.

Surface-porous triple-network hydrogels may have improved osseointegration with a porous layer as thin as about 0.4 mm thick while maintaining the majority of the strength and elastic modulus of the hydrogel.

### Method of Manufacture

In general, the triple-network hydrogels described herein may be fabricated in any appropriate manner. In one variation an initial scaffold (e.g., sheet, form, plug, etc.) of cross-linked fiber network may be infiltrated with the double-network hydrogel to from the triple-network hydrogel. The cross-linked fiber network may be formed of a variety of sheets of material, such as sheets of a network of bacterial cellulose (BC) or a network of bacterial cellulose and polyacrylamide (BC-PAAm) that may be compressed into a stack of the desired height (e.g., between about 2 mm to 10 mm), and infiltrated with a double-network hydrogel, such as a PAMPS-PDMAAm double-network hydrogel or PAMPS-PAAm hydrogel to a final weight % of the BC r BC-PAAm of between about 2-25 weight % (e.g., between about 2-20 weight %, etc.).

For example, a piece of bacterial cellulose sheet is soaked in a solution of 2-acrylamido-2-methylpropanesulfonic acid (AMPS), cross linker (e.g., MBAA) and 0.5 w/v % 2-hydroxy-1-[4-(2-hydroxyethoxy) phenyl]-2-methyl-1-propanone (12959) overnight. The concentration of AMPS and MBAA can be varied, e.g., as shown in FIG. 9, to change the stiffness and strength of the hydrogel. Then, the bacterial cellulose may be pressed into a mold to give it a desired shape and size. The bacterial cellulose may then be cured with UV light for 15 minutes under constant pressure in the mold. The concentration of bacterial cellulose in the final product may be controlled by controlling the thickness to which the original bacterial cellulose sheet was compressed. The effect of changing the bacterial cellulose concentration is shown in FIGS. 4A, 5A, 6A and 6B. After UV curing, the bacterial cellulose-PAMPS hydrogel is soaked in a solution of acrylamide, 2 mM MBAA and 0.5 w/v % 12959 overnight. The concentration of acrylamide can be varied, e.g., within the range shown in FIG. 9, to adjust the stiffness and strength of the hydrogel. After soaking, the cellulose-PAMPS hydrogel will be taken out and cured with UV light again for 15 minutes. The time for both UV curing steps may vary according to the thickness of the hydrogel.

### Methods of Use of Triple-Network Hydrogels for Cartilage Repair or Replacement

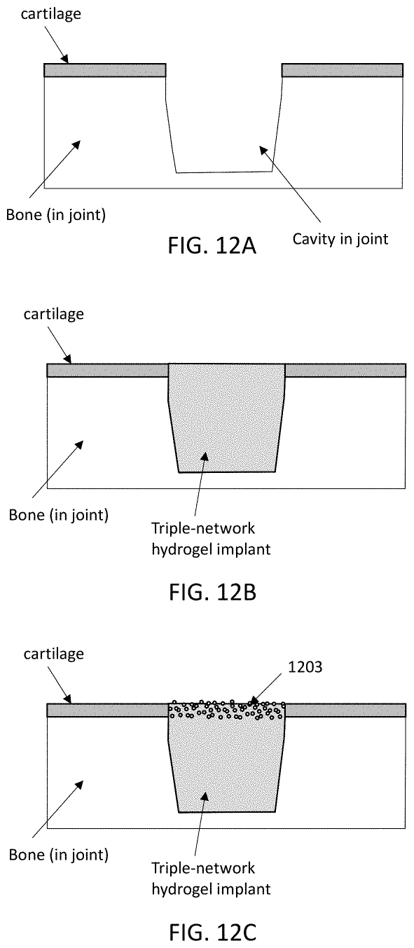

Another method for the use of a triple-network hydrogel implant as described herein is through the filling of a cavity in a joint. The cavity can be an existing one or one that is prepared by a surgeon. A triple-network hydrogel implant can be configured as a plug that can be inserted into a cavity. FIGS. 12A-12C shows an example of a cavity (e.g., FIG. 12A) filled with a hydrogel plug (FIG. 12B), including, in some variations, a triple-network hydrogel plug including pores, such as a porous outer region 1203, such as shown in FIG. 12C. The triple-network hydrogel plug can be of any shape and size; for instance it can be cylindrical in shape, tapered, etc. In some embodiments the triple-network hydrogel plug can be oversized to be elevated from the surrounding cartilage surface. In other embodiments the plug can be undersized to stay recessed in the cavity. The over-sizing or under-sizing can be such that the triple-network hydrogel plug can stand proud above the surrounding cartilage surface or recessed from the surrounding cartilage surface by about less than 1 mm, by about 1 mm, by more than about 1 mm, by about 2 mm, by about 3 mm, or by about more than 3 mm. In some embodiments the hydrogel plug can be slightly dehydrated to shrink its size and to allow an easy placement into the cavity. The hydrogel plug then can be hydrated and swollen in situ to cause a better fit into the cavity. The dehydrated and re-hydrated dimensions of the hydrogel plug can be tailored to obtain a good fit, under-sizing, or over-sizing of the plug after re-dehydration and re-swelling. The re-dehydration in situ can also be used to increase the friction fit between the plug and the cavity. This can be achieved by tailoring the dimensions and the extent of dehydration such that upon re-dehydration the cross-section of the plug can be larger than the cross-section of the cavity; by for instance about 1 mm, less than 1 mm, or more than 1 mm. In some embodiments the cavity is filled with an injectable form of the triple-network hydrogel material described herein.

Dehydration of the triple-network hydrogels described herein may be achieved by a variety of methods. For instance, a triple-network hydrogel can be placed in vacuum at room temperature or at elevated temperatures to drive out the water and cause dehydration. The amount of vacuum can be reduced by adding air or inert gas to the vacuum chamber where the triple-network hydrogel is placed during dehydration. Dehydration of the triple-network hydrogel also can be achieved by keeping it in air or inert gas at room temperature or at an elevated temperature. Dehydration in air or inert gas also can be carried out at temperatures lower than room temperature. Dehydration of the triple-network hydrogel may also be carried out by placing the hydrogel in a solvent. The solvent may drive water out of the hydrogel. Solvent dehydration also can be carried out at elevated temperatures. These dehydration methods can be used in combination with each other. Re-hydration of the triple-network hydrogel can be done in water containing solutions such as, saline, water, deionized water, salinated water, or an aqueous solution or DMSO.

The triple-network hydrogels described herein may be shaped into a medical device and subsequently dehydrated. The dehydrated implant may then re-hydrated. The initial size and shape of the medical implant may be tailored such that the shrinkage caused by the dehydration and the swelling caused by the subsequent re-hydration may result in the desired implant size and shape that can be used in a human joint. For example, the starting shape of the triple-network hydrogel before deformation can be a rectangular prism, a cylinder, a cube, or a non-uniform shape.

The implants described herein can be used to treat osteoarthritis, rheumatoid arthritis, other inflammatory diseases, generalized joint pain, joints damaged in an accident, joints damaged while participating in athletics, joints damaged due to repetitive use, and/or other joint diseases. The various devices, systems, methods, and other features of the embodiments disclosed herein may be utilized or applied to other types of apparatuses, systems, procedures, and/or methods, including arrangements that have non-medical benefits or applications.

The triple-network hydrogel implants described herein may be any appropriate shape, including a cylindrical plug, or any other shape. For example, an upper surface of the implant may be contoured to abut particular anatomy (e.g., planar (e.g., flat), non-planar (e.g., curved, concave, convex, undulating, fluted)). The implant can include a generally circular, oval, rectangular, triangular, hexagonal, etc. cross-sectional shape, or irregular, and/or the like. In some embodiments, the implant is generally shaped like a cylinder or a mushroom. The overall shape of any of the implants disclosed herein can vary depending on the specific application or use.

The shape may be formed by a molding process, a cutting process, or the like.

The triple-network hydrogel implants described herein may be customized to the patient. For example, any of these implants may be designed or customized for a specific subject's anatomy. For example, a surface of a bone and/or an opposing bone may be scanned (e.g., via computerized tomography (CT), computerized axial tomography (CAT), positron emission tomography (PET), magnetic resonance imaging (MRI), combinations thereof, etc.), which can be used to make a mold (e.g., via 3D printing, CAD-CAM milling, etc.) to match specific anatomical features of a specific patient or subject. Thus, one or more surfaces of the triple-network hydrogel implant may be customized to a certain shape. For another example, the bottom of the implant may be customized such that one or more outer surfaces of the triple-network hydrogel takes a certain shape. A custom implant can be advantageous, for example, when the anatomy has been damaged or otherwise includes unique characteristics. Alternatively, a generic implant (or implants having ranges of sizes) may be provided and cut or trimmed to fit.

In some embodiments, a scan may reveal that a plurality of implants may be used for treatment. For example, compared to one implant, a plurality of implants may be better able to treat a large defect, be better provide a load bearing surface to key points, and/or provide better access to a physician. The scan can be used to select locations and/or sizes for a plurality of implants. For example, taking a knee joint as an example, a user may select in a scan a portion of a lateral condyle for a first implant and a portion of a medial condyle for a second implant. If the implant would provide an advantage if the portion is a little more anterior, a little more posterior, a little more medial, a little more lateral, etc., the implant can be customized to that particular location using the scan, which may result in, for example, different load bearing surface features, different dimensions, different protrusion amounts, combinations thereof, and the like.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject is in need of cartilage repair or replacement.

Unless otherwise defined, all technical terms used herein have the same meaning as commonly understood by one of ordinary skill in the art to which this disclosure belongs.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Thus, throughout this specification and the claims which follow, unless the context requires otherwise, the word “comprise”, and variations such as “comprises” and “comprising” means various components can be co-jointly employed in the methods and articles (e.g., compositions and apparatuses). For example, the term “comprising” will be understood to imply the inclusion of any stated elements or steps but not the exclusion of any other elements or steps. The use herein of the terms “including,” “comprising,” or “having,” and variations thereof, are meant to encompass the elements listed thereafter and equivalents thereof as well as additional elements. Embodiments recited as “including,” “comprising” or “having” certain elements are also contemplated as “consisting essentially of” and “consisting of” those certain elements. Thus, in general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An artificial cartilage material comprising a triple-network hydrogel including:
a cross-linked cellulose nanofiber network having a tensile strength of greater than 5 MPa and a tensile modulus of greater than 8 MPa; and
a double network hydrogel having a compression strength of greater than 14 MPa, wherein the cross-linked nanofiber network is between 2-25 weight % of the triple-network hydrogel.

2. The artificial cartilage material of claim 1, further comprising an outer region having a porosity of between 0.1-300 micrometers diameter.

3. The artificial cartilage material of claim 2, wherein the outer region has a thickness of between 0.1 and 2.5 mm.

4. The artificial cartilage material of claim 2, further comprising a coating on the outer region of one or more of: hydroxyapatite (HA) and insulin-like growth factor I (IGF).

5. The artificial cartilage material of claim 1, wherein the cross-linked cellulose nanofiber network comprises bacterial cellulose having a tensile modulus of greater than 8 MPa.

6. The artificial cartilage material of claim 1, wherein the triple-network hydrogel has a tensile strength of between 4-10 MPa, a tensile modulus of between 8-25 MPa, a compression strength of between 14-60 MPa, and a compression modulus of between 8-22 MPa.

7. The artificial cartilage material of claim 1, wherein the triple-network hydrogel has a coefficient of friction of less than 0.1 at 1 mm/sec.

8. The artificial cartilage material of claim 1, wherein the double network hydrogel includes a polfacrylamide-methyl propyl sulfonic acid (PAMPS).

9. The artificial cartilage material of claim 1, wherein the double network hydrogel includes a polfacrylamide-methyl propyl sulfonic acid (PAMPS) and one or more of: polyacrylamide (PAAm) and poly-(N,N′-dimethyl acrylamide) (PDMAAm).

10. The artificial cartilage material of claim 1, a body forming a plug of the triple-network hydrogel.

11. An artificial cartilage material comprising a triple-network hydrogel including:
a cross-linked bacterial cellulose nanofiber network having a tensile strength of greater than 5 MPa and a tensile modulus of greater than 8 MPa; and
a negatively charged double network hydrogel including polfacrylamide-methyl propyl sulfonic acid and having a compression strength of greater than 14 MPa, wherein the cross-linked nanofiber network is between 2-25 weight % of the triple-network hydrogel.

12. The artificial cartilage material of claim 11, further comprising an outer region having a porosity of between 0.1-300 micrometers diameter.

13. The artificial cartilage material of claim 12, wherein the outer region has a thickness of between 0.1 and 2.5 mm.

14. The artificial cartilage material of claim 12, further comprising a coating on the outer region of one or more of: hydroxyapatite (HA) and insulin-like growth factor I (IGF).

15. The artificial cartilage material of claim 11, wherein the triple-network hydrogel has a tensile strength of between 4-10 MPa, a tensile modulus of between 8-25 MPa, a compression strength of between 14-60 MPa, and a compression modulus of between 8-22 MPa.

16. The artificial cartilage material of claim 11, wherein the triple-network hydrogel has a coefficient of friction of less than 0.1 at 1 mm/sec.

17. The artificial cartilage material of claim 11, wherein the double network hydrogel includes the polfacrylamide-methyl propyl sulfonic acid (PAMPS) and one or more of: polyacrylamide (PAAm) and poly-(N,N′-dimethyl acrylamide) (PDMAAm).

18. The artificial cartilage material of claim 11, a body forming a plug of the triple-network hydrogel.

19. An artificial cartilage material comprising a triple-network hydrogel including:
a cross-linked nanofiber network having a tensile strength of greater than 5 MPa; and
a double network hydrogel having a compression strength of greater than 14 MPa, wherein the cross-linked nanofiber network is between 2-25 weight % of the triple-network hydrogel.
